(19) United States
(12) Patent Application Publication
(10) Pub. No.: US 2023/0165912 A1
(43) Pub. Date: Jun. 1, 2023

(54) COMPOSITION FOR IMPROVING IMMUNITY

(71) Applicant: The Chinese University of Hong Kong, Shatin, New Territories (CN)

(72) Inventors: Siew Chien NG, Shatin,NewTerritories (CN); Ka Leung Francis CHAN, Tai Po, New (CN); Zhilu XU, Shijiazhuang Hebei (CN); Wingyan TANG, Hong Kong (CN); Qiaoyi LIANG, Tuen Mun, New Territories (CN)

(21) Appl. No.: 17/921,788

(22) PCT Filed: Apr. 28, 2021
(86) PCT No.: PCT/CN2021/090531

§ 371 Date: Oct. 27, 2022

Related U.S. Application Data
(60) Provisional application No. 63/016,759, filed on Apr. 28, 2020.
(60) Provisional application No. 63/025,310, filed on May 15, 2020.
(60) Provisional application No. 63/064,821, filed on Aug. 12, 2020.

(30) Foreign Application Priority Data
Jul. 9, 2020 (CN) 202010657312.5
Nov. 12, 2020 (CN) 202011259564.9
Mar. 1, 2021 (CN) 202110223880.9

(51) Int. Cl. A61K 35/741; A23L 33/135; A23L 33/21; A61K 35/745; A61K 35/747; A61P 1/00; A61P 37/02

(52) U.S. Cl. CPC A61K 35/741; A23L 33/21; A23L 33/135; A61K 35/745; A61K 35/747; A61P 1/00; A61P 37/02

(57) ABSTRACT

A probiotic composition comprising Bifidobacterium bifidum and Bifidobacterium longum, and may further comprising Bifidobacterium adolescentis and Lactobacillus rhamnosus is provided. A prebiotic composition comprising xylooligosaccharide, galactooligosaccharide, and corn dietary fiber, and a dietary composition comprising the probiotic composition and the prebiotic composition are provided. Use of the foregoing compositions in the preparation of a dietary product or a drug for assisting in preventing and/or treating a pathogen infection of an individual, enhancing the therapeutic effect of a pathogen infection of an individual, improving the immunity of an individual, or balancing the gut microecology of an individual is provided. The foregoing compositions can be used in patients suffering from COVID-19.

### RELATED APPLICATION

The present application claims priority to Chinese Patent Application Nos. 202010657312.5, 202011259564.9 and 202110223880.9, the entire contents of which are incorporated herein by reference. The present application also claims priority to U.S. Provisional Application No. 63/016,759, filed Apr. 28, 2020, U.S. Provisional Application No. 63/025,310, filed May 15, 2020, and U.S. Provisional Application No. 63/064,821, filed Aug. 12, 2020, the entire contents of which are incorporated herein by reference.

### TECHNICAL FIELD

The present application generally relates to the field of pharmaceuticals, food products, and health products. In particular, there is provided in the application a composition that improves the immunity of an individual or assists in treating and preventing a disease or improves the therapeutic effect of a disease based on regulation of gut flora and uses of the composition.

### BACKGROUND

There are various and complex bacteria in the gut flora of human or animal bodies, and there are many research reports on the gut flora. Gut flora is not only associated with digestive function, but also associated with the ability of the body to resist a disease, such as a pathogen infection, and an autoimmune disease, and even the response to drug therapy.

There is one class of bacteria in the gut flora that is beneficial to the body, known as probiotics. In general, probiotics refer to one class of beneficial active microorganisms to a host that colonize in humans to alter the composition of the flora in a certain site of the host. Probiotics can promote nutrient absorption and maintain the health of the intestine by regulating the immune function of the host mucosa and the system or by regulating the balance of the gut flora, thereby producing a beneficial health effect. Common probiotics include bifidobacteria, lactobacilli, and yeasts.

Some substances are closely related to probiotics and are referred to as prebiotics. In general, prebiotics refer to organic substances that are not digested and absorbed by a host but can selectively promote the metabolism and proliferation of probiotics in the body, thereby improving the health of the host. In general, prebiotics should be largely undigested and fermented by gut flora when they pass through the upper digestive tract. Most importantly, prebiotics can stimulate the growth of beneficial flora, but not stimulate harmful bacteria with potential pathogenicity or spoilage activity. Common prebiotics are oligosaccharides, also known as dietary fibers.

It has been a long-standing topic in this field to regulate gut flora based on probiotics/prebiotics, thereby promoting the health level of the body (e.g., improving immunity).

### SUMMARY OF THE INVENTION

In a first aspect, there is provided in the application a probiotic composition comprising Bifidobacterium bifidum and Bifidobacterium longum.

In some embodiments, the ratio of the amounts of Bifidobacterium bifidum and Bifidobacterium longum calculated by colony forming units is 1: (0.21-2.36).

In some embodiments, the probiotic composition further comprises Bifidobacterium adolescentis.

In some embodiments, the ratio of the amounts of Bifidobacterium adolescentis, Bifidobacterium bifidum, and Bifidobacterium longum calculated by colony forming units is (0.57-3.56): 1: (0.21-2.36). In some specific embodiments, the ratio of the amounts of Bifidobacterium adolescentis, Bifidobacterium bifidum, and Bifidobacterium longum calculated by colony forming units is (0.75-1): 1: (0.75-1).

In some embodiments, the probiotic composition further comprises Lactobacillus rhamnosus.

In some embodiments, the ratio of the amounts of Bifidobacterium adolescentis, Bifidobacterium bifidum, Bifidobacterium longum, Lactobacillus rhamnosus calculated by colony forming units is (0.57-3.36): 1: (0.21-2.36): 1.

In some embodiments, the probiotic composition is in unit dosage form, and the amounts of Bifidobacterium adolescentis, Bifidobacterium bifidum, Bifidobacterium longum, Lactobacillus rhamnosus calculated by colony forming units are independently in the order of 10⁴ to 10¹² CFU. In some embodiments, the total amount of Bifidobacterium adolescentis, Bifidobacterium bifidum, Bifidobacterium longum, Lactobacillus rhamnosus is in the order of 10⁶ to 10¹² CFU.

In some embodiments, the probiotic composition is for administration to an adult, and the amount of Bifidobacterium adolescentis is 2.59× 10⁵ - 4.49× 10¹¹ CFU; and/or the amount of Bifidobacterium bifidum is 1.26×10⁵ - 7.35×10¹¹ CFU; and/or the amount of Bifidobacterium longum is 2.23×10⁵ - 7.02×10¹¹ CFU; and/or the amount of Lactobacillus rhamnosus is 1.26×10⁵ - 2.59×10¹¹ CFU.

In some embodiments, the probiotic composition is for administration to a child, and the amount of Bifidobacterium adolescentis is 2.05×10⁵- 4.55×10¹¹ CFU; and/or the amount of Bifidobacterium bifidum is 1.47×10⁵-3.6×10¹¹ CFU; and/or the amount of Bifidobacterium longum is 7.55×10⁴-2.5×10¹¹ CFU; and/or the amount of Lactobacillus rhamnosus is 1.47× 10⁵- 3.6×10¹¹ CFU.

In some embodiments, the probiotic composition is free of probiotics other than the probiotics described in various embodiments of the present application. In some embodiments, the probiotic composition is free of probiotics other than Bifidobacterium adolescentis, Bifidobacterium bifidum, Bifidobacterium longum, and Lactobacillus rhamnosus. In some embodiments, the probiotic composition is free of Bifidobacteria other than Bifidobacterium adolescentis, Bifidobacterium bifidum, and Bifidobacterium longum.

In a second aspect, there is provided in the application a prebiotic composition comprising xylooligosaccharide, galacto-oligosaccharide, and corn dietary fiber.

In some embodiments, the ratio of the amounts of xylooligosaccharide, galactooligosaccharide, and corn dietary fiber by weight is (0.25-5): (0.75-4): (0.5-1). In some specific embodiments, the ratio of the amounts of xylooligosaccharide, galactooligosaccharide, and corn dietary fiber by weight is (0.25-0.5): (2-4): (0.5-0.75).

In some embodiments, the prebiotic composition is in unit dosage form, and the total amount of xylooligosaccharide, galactooligosaccharide, and corn dietary fiber by weight is 0.1-12 g. In some embodiments, the prebiotic composition is in unit dosage form, and the total amount of xylooligosaccharide, galactooligosaccharide, and corn dietary fiber by weight is 0.1-5 g.

In some embodiments, the amount of xylooligosaccharide is 0.01 g-6 g; and/or the amount of galactooligosaccharide is 0.04 g-9.6 g; and/or the amount of corn dietary fiber is 0.01 g-6 g.

In some embodiments, the prebiotic composition is free of prebiotic components other than xylooligosaccharide, galactooligosaccharide, and corn dietary fiber.

In a third aspect, there is provided in the application a dietary composition (in some cases, also referred to as a synbiotic composition) comprising the probiotic composition according to the first aspect and the prebiotic composition according to the second aspect.

In some specific embodiments, the dietary composition comprises Bifidobacterium adolescentis, Bifidobacterium bifidum, and Bifidobacterium longum as the probiotics and xylooligosaccharide, galactooligosaccharide, and corn dietary fiber as the prebiotics, wherein the ratio of the amounts of Bifidobacterium adolescentis, Bifidobacterium bifidum, and Bifidobacterium longum calculated by colony forming units (CFUs) is (0.75-1): 1: (0.75-1), and the total amount of the three bacteria is about 2×10¹¹CFU, and, the ratio of xylooligosaccharide, galactooligosaccharide and corn dietary fiber by weight is (0.25-0.5): (2-4): (0.5-0.75), and the total amount of xylooligosaccharide, galactooligosaccharide and corn dietary fiber is 1.2-1.5 g.

In some embodiments according to the first to third aspects, the probiotic composition or prebiotic composition or dietary composition is formulated for peroral administration. In some embodiments, the peroral administration comprises oral administration, mixing with oral products, and gavage.

In some embodiments according to the first to third aspects, the probiotic composition or prebiotic composition or dietary composition is a food supplement, a food additive, or a food product.

In some embodiments according to the first to third aspects, the probiotic composition or prebiotic composition or dietary composition is formulated as a powder, a granule, a tablet, or a capsule.

In some embodiments according to the first to third aspects, the probiotic composition or prebiotic composition or dietary composition is administered to a subj ect for assisting in preventing and/or treating a pathogen infection, or for enhancing the therapeutic effect of a pathogen infection, improving the immunity in the subject, or balancing the gut microecology in thesubject (including increasing microbial abundance, increasing desirable bacterial species, and/or reducing undesirable bacteria). In some embodiments, the pathogen is a virus, a bacterium, or a fungus. In some embodiments, the pathogen is a respiratory disease virus, such as COVID-19, an influenza virus, or respiratory syncytial virus.

In a fourth aspect, there is provided in the application use of the probiotic composition according to the first aspect, or the prebiotic composition according to the second aspect, or the dietary composition according to the third aspect, in the preparation of a dietary product or a drug for assisting in preventing and/or treating the pathogen infection in a subject, enhancing the therapeutic effect of a pathogen infection in a subject, or improving the immunity in a subject, or balancing the gut microecology in a subject (including increasing microbial abundance, increasing desirable bacterial species, and/or reducing undesirable bacteria). In some embodiments, the pathogen is a virus, a bacterium, or a fungus. In some embodiments, the pathogen is a respiratory disease virus, such as a novel coronavirus (COVID-19), an influenza virus, or a respiratory syncytial virus.

In a fifth aspect, there is provided in the application a method for assisting in preventing and/or treating the pathogen infection in a subject, enhancing the therapeutic effect of a pathogen infection in a subject, or improving the immunity in a subject, or balancing the gut microecology in a subject (including increasing microbial abundance, increasing desirable bacterial species, and/or reducing undesirable bacteria), comprising administering to the subject the probiotic composition according to the first aspect, or the prebiotic composition of the second aspect, or the dietary composition according to the third aspect. In some embodiments, the pathogen is a virus, a bacterium, or a fungus. In some embodiments, the pathogen is a respiratory disease virus, such as COVID-19, an influenza virus, or respiratory syncytial virus.

### BRIEF DESCRIPTION OF THE DRAWINGS

FIG. 1 shows the relationship between the desirable bacterial species and various probiotics identified in previous studies. desirable bacterial species have the potential to enhance immunity and their abundances are negatively correlated with COVID-19 disease severity or SARS-CoV-2 viral load. The circle represents a positive correlation between the abundance of the desirable bacterial species and the probiotics, the size indicates the strength of the positive correlation, and the box circles the target probiotics identified in the present application.

FIG. 2 shows partial results of the cohort studies in Example 2, wherein panel A shows the positive rates and the overall positive rates of Bifidobacterium adolescentis, Bifidobacterium bifidum, and Bifidobacterium longum in each cohort study; and panel B shows the incidence of three bifidobacteria, any two of the three bifidobacteria, or only one or none of the three bifidobacteria contained in the fecal samples of subjects.

FIG. 3 shows partial results of COVID-19 patients treated with the synbiotic composition and standard treatment in the clinical study of Example 3. Panel A shows clinical symptom relief scores at weeks 1 and 2 (the score is defined as 20) and the condition of antibodies form. Panel B shows the quantification of immune response markers in plasma (converted to log10). For all box plots, the centers are plotted by measured median, and upper and lower boundaries of the box plots correspond to the first and third percentiles, respectively. The p value is determined by two sides, and p value < 0.05 is considered statistically significant (Wilcoxon rank sum test). Panel C shows the percent decrease in inflammatory immune response markers at week 5 compared to baseline, with each bar representing the median percent decrease, and p values is determined by the two-sided Wilcoxon rank sum test. Panel D shows probiotic concentrations at baseline, and 2 weeks and 5 weeks after the subjects first took the synbiotic composition. The concentrations are determined by qPCR and shown after log10 transformation (ng/µl). P value < 0.05 is considered statistically significant (Wilcoxon rank sum test).

FIG. 4 shows the study scheme of Example 4.

FIG. 5 shows the total relative abundance of three bifidobacterium probiotics (Bifidobacterium adolescentis, Bifidobacterium bifidum, and Bifidobacterium longum, which are comprised in the synbiotic composition) in healthy persons and COVID-9 patients in the synbiotic composition group and the standard treatment group at baseline, weeks 2, 4, and 5 in Example 4, where the upper graph is a summary graph and the lower graphs are those at each time point. The relative abundance (percent) is shown as being converted to log10. The p value between the relative abundance at weeks 2, 4, and 5 and that at baseline is determined by Wilcoxon rank sum test.

FIG. 6 shows shannon diversity index of healthy persons and COVID-9 patients in the synbiotic composition group and the standard treatment group at baseline, weeks 2, 4, and 5 in Example 4. The p value between the shannon diversity index at weeks 2, 4, and 5 and that at baseline is determined by Wilcoxon rank sum test.

FIG. 7 shows the total relative abundance of the desirable bacterial species (A) and the undesirable bacterial species (B) in healthy persons and COVID-9 patients in the synbiotic composition group and the standard treatment group at baseline, weeks 2, 4, and 5 in Example 4, where panel A shows the total relative abundance of the desirable bacterial species (those with higher abundance in non-COVID-19 humans) and panel B shows the total relative abundance of the undesirable bacterial species (those with higher abundance in COVID-19 patients). The relative abundance (percent) is shown as being converted to log10. The p value between the relative abundance at weeks 2, 4, and 5 and that at baseline is determined by Wilcoxon rank sum test.

FIG. 8 shows species with different abundance at baseline, weeks 2, 4 and 5 between the synbiotic composition group and the standard treatment group in Example 4 (LDA>2, p < 0.05). The levels of many desirable bacterial species (marked by box) are significantly higher in the synbiotic composition group compared to the standard treatment group, whereas the levels of undesirable bacterial species (Klebsiella pneumoniae, Veillonella parvula, and Escherichia coli) in the synbiotic composition group are significantly lower than those in the standard treatment group. The corresponding relationship between chromaticity of squares and LDA values is as shown in the diagram. If the LDA value is positive, it means that the level of this species is significantly higher in the synbiotic composition treatment group, with a darker color representing a greater difference. If the LDA value is negative, it means that the level of this species is significantly higher in the standard treatment group, and a lighter color represents a greater difference.

### DETAILED DESCRIPTION OF THE INVENTION

The present inventors have conducted extensive and in-depth studies on gut flora, in particular probiotics, and have discovered that probiotics are significantly positively correlated with desired bacterial species that can enhance immunity and can promote the prevention and treatment of a pathogen infection (e.g., a respiratory infection). Based on the above findings, a probiotic/prebiotic/synbiotic composition is provided. It is expected that these compositions can effectively enhance the immunity of the body and can contribute to the prevention and/or treatment of a pathogen infection, e.g., a respiratory pathogen infection, such as a novel coronavirus (COVID-19), an influenza virus, or a respiratory syncytial virus.

Hereinafter, the contents of the present application will be further explained according to some specific embodiments. However, the exemplified specific embodiments are for illustrative purposes only and are not intended to limit the scope of the present application. Those skilled in the art will recognize that the specific feature in one of the following embodiments can be used in any other embodiment without departing from the spirit of the present application.

Unless otherwise stated, the terms in this application have the same meaning as commonly understood by those skilled in the art. All patent documents, academic papers, and other publications cited herein are incorporated herein by reference in their entirety.

It will be appreciated that the specific values given herein can not only be understood as individual numerical values, but also be considered as providing end values for a certain range, and can be combined with each other to provide other ranges. For example, when it is disclosed that the content of a certain component of a composition is 1, 2 or 3 g, it is equivalent to the disclosure that the content of the component can be 1-2 g, 1-3 g or 2-3 g.

In a first aspect, there is provided in the application a probiotic composition comprising Bifidobacterium bifidum and Bifidobacterium longum.

As used herein, “probiotic composition” refers to a composition in which a probiotic is used as an active ingredient, and does not exclude the presence of an auxiliary ingredient required for the cultivation, isolation and purification of the probiotic and/or an adjuvant ingredient for formulating the composition according to a desired purpose.

In some embodiments, the ratio of the amounts of Bifidobacterium bifidum and Bifidobacterium longum calculated by colony forming units is 1: (0.21-2.36).

Colony forming unit (CFU) is a common form in the field to characterize the amount of a microorganism. Unless otherwise specified, the amount of microorganism described in the application is calculated in colony forming unit.

For example, the ratio of the amounts of Bifidobacterium bifidum and Bifidobacterium longum can be 1: (0.21, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.36). In some embodiments, the composition is for administration to an adult, and the ratio of the amounts of Bifidobacterium bifidum and Bifidobacterium longum is 1: (0.36-2.36). In some embodiments, the composition is administration to a child, and the ratio of the amounts of Bifidobacterium bifidum and Bifidobacterium longum is 1: (0.21-1.7).

In some embodiments, the probiotic composition further comprises Bifidobacterium adolescentis.

In some embodiments, the ratio of the amounts of Bifidobacterium adolescentis, Bifidobacterium bifidum, and Bifidobacterium longum calculated by colony forming units is (0.57-3.56): 1: (0.21-2.36). In some embodiments, the ratio of the amounts of Bifidobacterium adolescentis, Bifidobacterium bifidum, and Bifidobacterium longum is (0.57, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.56): 1: (0.21, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.36). In some specific embodiments, the ratio of the amounts of Bifidobacterium adolescentis, Bifidobacterium bifidum, and Bifidobacterium longum calculated by colony forming units is (0.75-1): 1: (0.75-1). In some embodiments, the composition is for administration to an adult, and the ratio of the amounts of Bifidobacterium adolescentis, Bifidobacterium bifidum, and Bifidobacterium longum is (1-3.56): 1: (0.86-2.36). In some embodiments, the composition is for administration to a child, and the ratio of the amounts of Bifidobacterium adolescentis, Bifidobacterium bifidum, and Bifidobacterium longum is (0.57-3.09): 1: (0.21-1.7).

In some embodiments, the probiotic composition further comprises Lactobacillus rhamnosus. Short chain fatty acids (SCFAs, such as butyric acid and propionic acid) can affect the differentiation or function of T cells, macrophages and dendritic cells, which is significant for maintaining immune homeostasis. Lactobacillus rhamnosus can increase the production of SCFA in the gut, and a combination of Lactobacillus rhamnosus, Bifidobacterium bifidum and Bifidobacterium longum is expected to increase the total production of SCFA. Furthermore, it has been reported that oral administration of Lactobacillus rhamnosus can increase the content of Bacteroides and Freudenreichii in the gut.

In some embodiments, the ratio of the amounts of Bifidobacterium adolescentis, Bifidobacterium bifidum, Bifidobacterium longum, and Lactobacillus rhamnosus is (0.57-3.56): 1: (0.21-2.36): 1. In some embodiments, the ratio of the amounts of Bifidobacterium adolescentis, Bifidobacterium bifidum, Bifidobacterium longum, and Lactobacillus rhamnosus calculated by colony forming units is (0.57, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.56): 1: (0.21, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.36): 1. In some embodiments, the composition is for administration to an adult, and the ratio of the amounts of Bifidobacterium adolescentis, Bifidobacterium bifidum, Bifidobacterium longum, and Lactobacillus rhamnosus is (1-3.56): 1: (0.86-2.36): 1. In some embodiments, the composition is for administration for a child, and the ratio of the amounts of Bifidobacterium adolescentis, Bifidobacterium bifidum, Bifidobacterium longum, Lactobacillus rhamnosus is (0.57-3.09): 1: (0.21-1.7): 1.

In some embodiments, the probiotic composition is in unit dosage form, and the amounts of Bifidobacterium adolescentis, Bifidobacterium bifidum, Bifidobacterium longum, and Lactobacillus rhamnosus calculated by colony forming units are independently in the order of 10⁴ to 10¹² CFU. In some embodiments, the total amount of Bifidobacterium adolescentis, Bifidobacterium bifidum, Bifidobacterium longum, and Lactobacillus rhamnosus is in the order of 10⁶ to 10¹² CFU. In some specific embodiments, the total amount of Bifidobacterium adolescentis, Bifidobacterium bifidum, Bifidobacterium longum, and Lactobacillus rhamnosus is about 2×10¹¹CFU. It is to be understood that the probiotic compositions of the present application do not necessarily comprise all of Bifidobacterium adolescentis, Bifidobacterium bifidum, Bifidobacterium longum, and Lactobacillus rhamnosus, and therefore, “total amount” herein refers to the total amount of the four probiotics present in the probiotic composition.

As used herein, “unit dosage form” refers to a single administration dose of a composition, either individually or separately packaged, which may generally be present in a single tablet, capsule or powder/particle bag, etc. In some embodiments, for ease of administration, a unit dosage form is prepared as a composition containing daily dosage.

In some embodiments, the probiotic composition is for administration to an adult, and the amount of Bifidobacterium adolescentis is 2.59×10⁵ - 4.49×10¹¹ CFU; and/or the amount of Bifidobacterium bifidum is 1.26×10⁵ - 7.35×10¹¹ CFU; and/or the amount of Bifidobacterium longum is 2.23×10⁵ - 7.02×10¹¹ CFU; and/or the amount of Lactobacillus rhamnosus is 1.26×10⁵ - 2.59×10¹¹ CFU.

In some embodiments, the probiotic composition is for administration to a child, and the amount of Bifidobacterium adolescentis is 2.05×10⁵- 4.55×10¹¹ CFU; and/or the amount of Bifidobacterium bifidum is 1.47×10⁵- 3.6×10¹¹ CFU; and/or the amount of Bifidobacterium longum is 7.55×10⁴- 2.5×10¹¹ CFU; and/or the amount of Lactobacillus rhamnosus is 1.47×10⁵- 3.6×10¹¹ CFU.

In some embodiments, the probiotic composition is free of probiotics other than the probiotics described in various embodiments of the present application.

In the technical context of such embodiments, “free of” should be understood as “substantially free of”, which does not exclude the presence of minor or trace amounts of other probiotics due to factors such as cultivation, isolation, and purification of strains. In some embodiments, the amount of other probiotics is no more than 5%, preferably no more than 1% of the total amount of probiotics in the composition.

In some embodiments, the probiotic composition is free of probiotics other than Bifidobacterium adolescentis, Bifidobacterium bifidum, Bifidobacterium longum, and Lactobacillus rhamnosus. In some embodiments, the probiotic composition is free of Bifidobacteria other than Bifidobacterium adolescentis, Bifidobacterium bifidum, and Bifidobacterium longum.

In a second aspect, there is provided in the application a prebiotic composition comprising xylooligosaccharide, galactooligosaccharide, and corn dietary fiber.

As used herein, “prebiotic composition” refers to a composition in which a prebiotic is used as an active ingredient, and does not exclude the presence of an auxiliary ingredient introduced due to the synthesis, isolation, purification and the like of the prebiotic and/or an adjuvant ingredient for formulating the composition according to a desired purpose.

In some embodiments, the ratio of the amounts of xylooligosaccharide, galactooligosaccharide, and corn dietary fiber by weight is (0.25-5): (0.75-4): (0.5-1). In some embodiments, the ratio of the amounts of xylooligosaccharide, galactooligosaccharide, and corn dietary fiber by weight is (0.25, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0): (0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0): (0.5, 0.6, 0.7, 0.8, 0.9, 1.0). In some specific embodiments, the ratio of the amount of xylooligosaccharide, galactooligosaccharide and corn dietary fiber by weight is (0.25-0.5): (2- 4): (0.5-0.75).

In some embodiments, the prebiotic composition is in unit dosage form, and the total amount of xylooligosaccharide, galactooligosaccharide and corn dietary fiber by weight is 0.1-12 g, e.g., 0.1, 0.2, 0.3, 0.4, 0.5, 0.6, 0.7, 0.8, 0.9, 1, 2, 3, 4, 5, 6, 7, 8, 9, 10, 11, 12 g.

In some embodiments, the prebiotic composition is in unit dosage form, and the amount of xylooligosaccharide is 0.01 g-6g; and/or the amount of galactooligosaccharide is 0.04 g-9.6 g; and/or the amount of corn dietary fiber is 0.01 g-6 g. Such unit dosage forms can be administered to adults or children.

In some embodiments, when the prebiotic composition is used as a food supplement or additive, the total amount of xylooligosaccharide, galactooligosaccharide and corn dietary fiber can be controlled to 0.1-5 g, and the total amount after collocation with the food is expected to reach a desired value, such as about 12 g.

In some embodiments, the prebiotic composition is free of prebiotic components other than xylooligosaccharide, galactooligosaccharide and corn dietary fiber.

In the technical context of such embodiments, “free of” should be understood as “substantially free of”, which does not exclude the presence of minor or trace amounts of other prebiotics due to the factors such as synthesis, extraction, isolation, and purification of the prebiotic. In some specific embodiments, the amount of other prebiotics is no more than 5%, preferably no more than 1% of the total amounts of prebiotics in the composition.

In a third aspect, there is provided in the application a dietary composition (sometimes also referred to as “a synbiotic composition”) comprising the probiotic composition according to the first aspect and the prebiotic composition according to the second aspect.

It will be appreciated by those skilled in the art that the above dietary composition need not be obtained by separately preparing the probiotic composition according to the first aspect and the prebiotic composition according to the second aspect, and then mixing or compounding the above two compositions. As long as one dietary composition encompasses all features of one embodiment of the probiotic composition according to the first aspect and all features of one embodiment of the prebiotic composition according to the second aspect, the dietary composition belongs to the composition of the third aspect according to the present application.

As one non-limiting example, the dietary composition of the present application can have the following formulation (daily dosage, which can be provided in unit dosage form):

| | | |
|---|---|---|
| Probiotic | Adults | Children |
| Bifidobacterium adolescentis | 2.59×10⁵-4.49×10¹¹ CFU | 2.05×10⁵-4.55×10¹¹ CFU |
| Bifidobacterium bifidum | 1.26×10⁵-7.35 × 10¹¹ CFU | 1.47×10⁵-3.6×10¹¹ CFU |
| Bifidobacterium longum | 2.23×10⁵ - 7.02×10¹¹ CFU | 7.55×10⁴-2.5×10¹¹ CFU |
| Lactobacillus rhamnosus | 1.26×10⁵ - 2.59×10¹¹ CFU | 1.47× 10⁵- 3.6×10¹¹ CFU |
| Xylooligosaccharide | 0.01 g-6g | |
| Galactooligosaccharide | 0.04 g-9.6 g | |
| Corn dietary fiber | 0.05-6 g | |

In some specific embodiments, the dietary composition comprises Bifidobacterium adolescentis, Bifidobacterium bifidum, and Bifidobacterium longum as the probiotics; and xylooligosaccharide, galactooligosaccharide, and corn dietary fiber as the prebiotics, wherein the ratio of the amounts of Bifidobacterium adolescentis, Bifidobacterium bifidum, and Bifidobacterium longum calculated by colony forming units (CFUs) is (0.75-1): 1: (0.75-1), and the total amount of the three bacteria is about 2×10¹¹CFU; and the ratio of xylooligosaccharide, galactooligosaccharides and corn dietary fiber by weight is (0.25-0.5): (2-4): (0.5-0.75), and the total amount of xylooligosaccharide, galactooligosaccharide and corn dietary fiber is 1.2-1.5 g.

In some embodiments according to the first to third aspects, the probiotic composition or prebiotic composition or dietary composition is formulated for peroral administration. In some embodiments, the peroral administration comprises oral administration, mixing with oral products, and gavage.

In some embodiments according to the first to third aspects, the probiotic composition or prebiotic composition or dietary composition is a food supplement, a food additive, or a food.

In some embodiments according to the first to third aspects, the probiotic composition or prebiotic composition or dietary composition is formulatedinto a powder, a granule, a tablet, or a capsule.

The main administration mode of the probiotic composition or prebiotic composition or dietary composition of the present application is for administration to the gastrogut of an individual. Direct peroral administration is convenient, but for certain special individuals, such as bedridden patients, the probiotic composition can also be administrated by gavage or the like.

The product form of the probiotic composition or prebiotic composition or dietary composition of the present application can be varied. For example, it can be prepared as a separate dietary supplement (e.g., a capsule, a tablet, a powder, or a granule) to be taken with or without meals. It can be prepared into additive type products, such as various solid/semi-solid foods, blended powder/granular foods, beverages, which can be added or formulated prior to ingestion by an individual. It can also be used as a direct component of various solid/semi-solid foods, blended powder/granulated foods, and beverages.

In some embodiments according to the first to third aspects, the probiotic composition or prebiotic composition or dietary composition is administered to a subject for assisting in preventing and/or treating a pathogen infection, or for enhancing the therapeutic effect of a pathogen infection, improving the immunity in the subject, or balancing the gut microecology in the subject (including increasing microbial abundance, increasing desirable bacterial species, and/or reducing undesirable bacteria). In some embodiments, the pathogen is a virus, a bacterium, or a fungus. In some embodiments, the pathogen is a respiratory disease virus, such as COVID-19, an influenza virus, or respiratory syncytial virus.

In a fourth aspect, there is provided in the application use of the probiotic composition of the first aspect, or the prebiotic composition of the second aspect, or the dietary composition of the third aspect, in the preparation of a dietary product or a drug for assisting in preventing and/or treating the pathogen infection in a subject, or enhancing the therapeutic effect of a pathogen infection in a subject, or improving the immunity in a subject, or balancing the gut microecology in a subject (including increasing microbial abundance, increasing desirable bacterial species, and/or reducing undesirable bacteria). In some embodiments, the pathogen is a virus, a bacterium, or a fungus. In some embodiments, the pathogen is a respiratory disease virus, such as COVID-19, an influenza virus, or a respiratory syncytial virus.

In the absence of conflict, the probiotic composition or prebiotic composition or dietary composition of the present application can be prepared by referring to the conventional processing modalities for probiotic or prebiotic products in the art. For example, various prebiotic or prebiotic ingredients can be mixed into the product either sequentially or simultaneously or as a lyophilized premix by conventional processing techniques.

In a fifth aspect, there is provided in the application a method for assisting in preventing and/or treating the pathogen infection in a subject, or enhancing the therapeutic effect of a pathogen infection in a subject, or improving the immunity in a subject, or balancing the gut microecology in a subject (including increasing microbial abundance, increasing desirable bacterial species, and/or reducing undesirable bacteria), comprising administering to the subject the probiotic composition according to the first aspect, or the prebiotic composition according to the second aspect, or the dietary composition according tothe third aspect. In some embodiments, the pathogen is a virus, a bacterium, or a fungus. In some embodiments, the pathogen is a respiratory disease virus, such as COVID-19, an influenza virus, or respiratory syncytial virus.

### EXAMPLES

The following examples are only for the purpose of illustration and not limiting of the scope of the present application.

### Example 1

This Example describes a first phase of cohort studies conducted by the inventors.

### Method

### Study Cohort 1

The inventors recruited 942 healthy Chinese from Hongkong (n = 61) and Yunan Province (n = 881). This study was approved by the Joint Chinese University of Hong Kong-New Territories East Cluster Clinical Research Ethics Committee (The Joint CUHK-NTEC CREC, CREC No.: 2016.407) and the Research Ethics Committee of the First Affiliated Hospital of Kunming Medical College (No. 2017.L. 14). All subjects signed written informed consent. Fecal samples of the subjects were stored at -80° C. for analysis on bacteriome.

### Study Cohort 2

The inventors publicly recruited 546 healthy Hong Kong adults. This study was approved by the Joint Chinese University of Hong Kong-New Territories East Cluster Clinical Research Ethics Committee (The Joint CUHK-NTEC CREC, CREC No.: 2016.707). All subjects signed written informed consent, agreed to donate fetal samples, and provided demographic information by a questionnaire survey. Fecal samples of the subjects were stored at -80° C. for analysis on bacteriome.

### Study Cohort 3

The inventors recruited 64 healthy children. This study was approved by the Joint Chinese University of Hong Kong-New Territories East Cluster Clinical Research Ethics Committee (The Joint CUHK-NTEC CREC, CREC No.: 2016.607). All subjects signed written informed consent, agreed to donate fetal samples, and provided demographic information by a questionnaire survey. Fecal samples of the subjects were stored at -80° C. for analysis on bacteriome.

### Fecal DNA Extraction and DNA Sequencing for Cohorts 1 and 3

Fecal DNA was extracted by using the Maxwell® RSC PureFood GMO and Authentication Kit (Promega). About 100 mg of each fecal sample was pre-washed with 1 ml ddH₂O and centrifuged at 13,000 g for 1 minute. The precipitate was resuspended in 800 µL TE buffer (pH 7.5), 1.6 µl 2-mercaptoethanol and 500 U lyase (Sigma) were added and the mixture was incubated at 37° C. for 60 minutes. The sample was then centrifuged at 13,000 g for 2 minutes and the supernatant was discarded. After pretreatment, DNA was subsequently extracted by using the Maxwell® RSC PureFood GMO and Authentication Kit (Promega) according to the product instructions. 1 ml CTAB buffer was added to the precipitate and shaken for 30 s, and then the solution was heated at 95° C. for 5 minutes. Thereafter, the sample was ground with beads (Biospec, 0.5 mm for fungi and 0.1 mm for bacteria, 1:1) under high-speed shaking for 15 minutes. Thereafter, 40 µl proteinase K and 20 µl RNA enzyme were added and incubated at 70° C. for 10 minutes. The supernatant was then obtained by centrifugation at 13,000 g for 5 minutes and placed in Maxwell®RSC instrument for DNA extraction. The extracted fecal DNA was subjected to ultra-deep metagenomic sequencing by Ilumina Novoseq 6000 (Novogen, Beijing, China). An average of 12G data was obtained for each sample.

### Fecal DNA Extraction and DNA Sequencing for Cohort 2

Fecal DNA was extracted by using the DNeasy PowerSoil Kit (QIAGEN) according to the manufacturer’s instructions. 0.1 g fecal sample was taken for DNA extraction, and then the extracted DNA concentration was determined by using the Qubit dsDNA BR Kit (Thermo Fisher Scientific). The DNA sample was sent to the sequencing service provider (Novogene HK Company Limited, Wanchai, Hong Kong) for library preparation and paired shotgun metagenomic sequencing (Illumina NovaSeq 6000), with each sample returning an average of 7.5 GB of raw data.

### Correlation Analysis Among Bacterial Species

Based on the previous study results of the inventors’ team of the present application, a number of bacteria in the gut, including a number of bacteroides species and Bifidobacterium pseudocatenulatum, are negatively correlated with disease severity or SARS-CoV-2 viral load in COVID-19 patients, suggesting that these species (also referred to herein as “desirable bacterial species”) have a protective effect on COVID-19 (related studies are described in U.S. Provisional Pat. Applications 63/016,759 and 63/025,310, the disclosures of which are incorporated herein by reference in their entirety for all purposes). However, most of the desirable bacterial species have not yet been approved for application in foods. Therefore, the inventors performed a correlation analysis between the species currently approved for food use and the amounts of the desirable bacterial species in the population. The desirable bacterial species for correlation analysis include Akkermansia muciniphila, Alistipes onderdonkii, Anaerostipes hadrus, Bacteroides dorei, Bacteroides massiliensis, Bacteroides ovatus, Bacteroides thetaiotaomicron, Bifidobacterium pseudocatenulatum, Eubacterium limosum, Eubacterium rectale, Eubacterium ventriosum, Faecalibacterium prausnitzii, Roseburia hominis, Roseburia intestinalis, and Eubacterium hallii.

Correlation analysis was conducted in study cohorts 1 and 2 in the following manner: Quality filtering and trimming of metagenomic readings were performed by using Trimmomatic (v0.38) default parameters. Then, the host DNA (reference genome: hg38) was removed by Kneaddata (v0.7.2 , https: //bitbucket.org/biobakery/kneaddata/wiki/Home). Species-level metagenomic annotation was performed by using MetaPhlAn26 (v2.6.0). The relative abundance generated by MetaPhlAn2 was then subjected to a Centered log ratio (clr). The Pearson correlation coefficient was calculated and plotted by using the R package corrplot v0.78.

### Results

### Identification of Probiotic Species

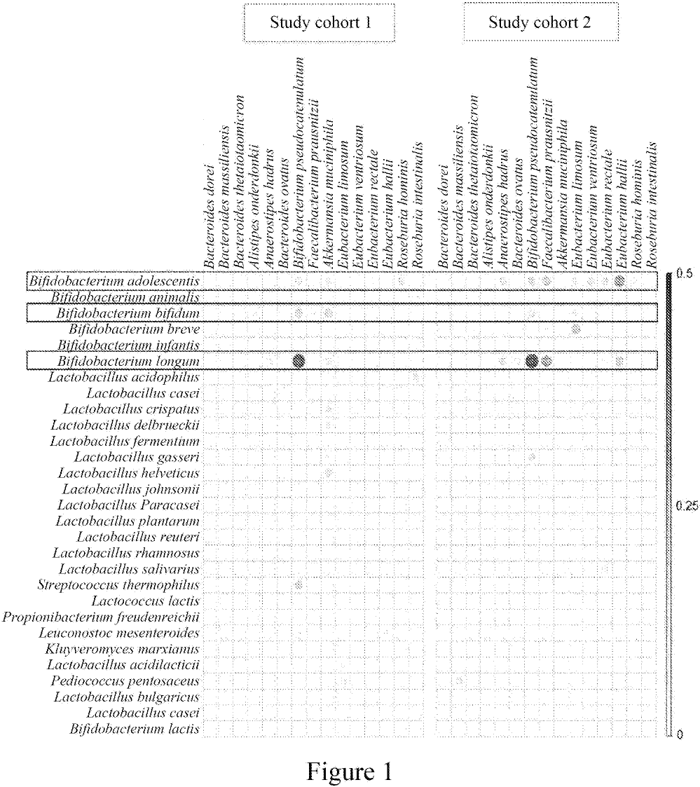

In the correlation analysis of study cohorts 1 and 2, the relative abundance of some probiotics was significantly correlated with the desirable bacterial species, among which, Bifidobacterium bifidum, Bifidobacterium longum and Bifidobacterium adolescentis were positively correlated with many desirable bacterial species (FIG. 1 and Table 1), suggesting that supplementation of these three probiotics can increase the content of at least a portion of the desirable bacterial species in the gut, improve gut health, thereby reducing infection risk or disease severity, such as enhancing immunity, and preventing and treating a respiratory infection.

TABLE 1

| Bacterial species | NCBI:txid |
|---|---|
| Bifidobacterium adolescentis | 1680 |
| Bifidobacterium bifidum | 1681 |
| Bifidobacterium longum | 216816 |

### Proportions of Probiotics

The inventors calculated the mean relative abundance of Bifidobacterium adolescentis, Bifidobacterium bifidum and Bifidobacterium longum in adults and children of study cohorts 1-3. The proportion of these probiotics was based on the natural proportion of various probiotics in healthy population, and was relatively fixed in the approximately 1500 healthy Chinese population of study cohort 1-3. Therefore, mimicking the proportion of bacterial species in healthy population might increase the chance of colonization of bacterial species in the gut. On this basis, the proportions of Bifidobacterium adolescentis, Bifidobacterium bifidum and Bifidobacterium longum in synbiotics were designed to be (1-3.56): 1: (0.86-2.36) for adults, and (0.57-3.09):1:(0.21-1.7):1 for children, respectively.

### Selection and Proportion of Prebiotics

Prebiotics and probiotics are generally complementary to each other, and therefore it is advantageous to add prebiotics to probiotics so as to be formulated into synbiotics after identification and selection of the probiotics. For the probiotic species identified in the above studies, the prebiotics selected by the inventors include xylooligosaccharide, galactooligosaccharide, and corn dietary fiber.

Xylooligosaccharide, also known as xylo-oligosacc, refers to a functional oligosaccharide in which 2 to 10 xylose molecules are linked by β-1,4 glycosidic linkages. Xylooligosaccharide is an excellent proliferation factor of Bifidobacterium. Xylooligosaccharide has obvious proliferationeffect on Bifidobacterium bifidum and Bifidobacterium adolescentis. It has been reported that Bifidobacterium adolescentis, Bifidobacterium infantis and Bifidobacterium bifidum all utilize xylooligosaccharide by producing xylosidase and arabinosidase, and their ability to hydrolyze xylooligosaccharide depends on the efficiency of their xylanase enzymolysis systems. The proliferation effect of xylooligosaccharide on Bifidobacterium and the yield of short chain fatty acids after fermentation decrease with the increase of molecular weight of xylooligosaccharide component. The digestion test in vitro showed that after passing through the saliva to the gut mucosa enzyme liquid, the residual rate of xylooligosaccharide reached 99.6%, which could be sufficiently fermented by Bifidobacterium in large intestine, and the proliferation effect of xylooligosaccharide on Bifidobacterium was 10 to 20 times that of other functional oligosaccharides. Xylooligosaccharide has the characteristics of acid resistance, high temperature resistance, strong stability, good compatibility and the like, and can be well applied to foods.

Galactooligosaccharide and corn dietary fiber can promote the growth of a variety of bifidobacteria. Galactooligosaccharide is a new type of functional substance and its molecular structure is generally that 1 to 7 galactosyl groups are linked to galactose or glucose molecules. It is one of the functional oligosaccharides with natural properties. It has good palatability, water solubility and stability, and can proliferate probiotics, especially Bifidobacterium, in the human gut after entering the human body, and meanwhile it can also inhibit the growth of spoilage bacteria. Probiotics in the gut can produce a large amount of exopolysaccharides while utilizing galactooligosaccharide to proliferate. Exopolysaccharides not only have anti-tumor activity, and immune activity, but also can promote the long-term colonization of probiotics in the gut. After entering the stomach, corn dietary fiber can absorb some water and promote gut to accelerate peristalsis, and accelerate fecal excretion, thereby reducing rectal pressure, and preventing and reducing gut disease. Meanwhile, Bifidobacterium has a fermentation effect on corn dietary fiber, which can be rapidly fermented by microorganisms in the cecum to produce short chain fatty acids.

According to the proportion of the probiotics, the inventors further provide a suitable proportion of the three prebiotics, wherein the proportion of xylooligosaccharide, galactooligosaccharide and corn dietary fiber can be (0.25-5): (0.75-4): (0.5-1).

### Example 2

This example describes an extended second phase of cohort studies conducted by the inventors based on the study cohort 2 in Example 1.

### Method

### Study Cohort HC (i.e., Study Cohort 2 in Example 1)

For fetal samples of the subjects, fetal DNA was extracted by using the QIAamp DNeasy PowerSoil Kit according to the manufacturer’s instructions.

### Study Cohort CR1

The inventors publicly recruited 219 healthy Hong Kong adults. This study was approved by the Joint Chinese University of Hong Kong-New Territories East Cluster Clinical Research Ethics Committee (The Joint CUHK-NTEC CREC, CREC No.: 2017.369). All subjects signed written informed consent, agreed to donate fetal samples, and provided demographic information by a questionnaire survey. Fecal samples of the subjects were stored at -80° C. for analysis on bacteriome. Fetal DNA was extracted by using the QIAamp DNeasy PowerSoil Kit according to the manufacturer’s instructions.

### Study Cohort CR2

The inventors randomly sampled fecal samples of 30 healthy Hong Kong adults from a group of asymptomatic subjects who underwent colonoscopy and the results were normal. This study was approved by the Joint Chinese University of Hong Kong-New Territories East Cluster Clinical Research Ethics Committee (The Joint CUHK-NTEC CREC, CREC No.: 2017.198). All subjects signed written informed consent, and agreed to donate fetal samples. Fecal samples of the subjects were stored at -80° C. prior to microbiome analysis. Fetal DNA was extracted by using the Maxwell RSC PureFood GMO and Authentication Kit according to the manufacturer’s instructions.

### Study Cohort Cov

Fecal samples from 78 healthy Hong Kong adults from a healthy control group of one COVID-19 study were included by the inventors. Fetal DNA was extracted by using the Maxwell RSC PureFood GMO and Authentication Kit according to the manufacturer’s instructions.

### Study Cohort LeanHC

The inventors randomly selected 67 healthy Hong Kong adults with BMI <23 from the study cohort HC. Fecal samples of the subjects were stored at -80° C. for microbiome analysis. Fetal DNA was extracted by using the Maxwell RSC PureFood GMO and Authentication Kit according to the manufacturer’s instructions.

### Fecal Samples and DNA Extraction

This example included fecal samples of healthy subjects collected from the five independent cohort studies. Fecal DNA was extracted by using QIAamp DNeasy PowerSoil Kit, Maxwell RSC PureFood GMO and Authentication Kit, or QIAamp DNA Stool Mini Kit according to the manufacturer’s instructions. The quality and quantity of DNA were determined by using a NanoDrop spectrophotometer and gel electrophoresis, respectively.

### Meta-Genome Sequencing

A DNA library was constructed by end repair, purification and PCR amplification procedures. After constructing the DNA library, the DNA library was sequenced on the NextSeq platform in the inventors’ laboratory using the 150 bp paired-end sequencing strategy. An average of 12 Gb of data per sample was available for further analysis. All experimental procedures were in accordance with the uniform standards of the inventors’ laboratory.

### Data Processing and Statistical Analysis of Metagenomic Data

The inventors used Fastp to perform mass filtration, PolyG tail modification and adapter modification on the sequenced fragments of metagenome, and the sequenced fragments of 50 bases or less were deleted. Next, the human genes in the sequenced fragments of the quality-trimmed metagenome were removed with KneadData, and then the metagenome at species level was analyzed with MetaPhlAn 2. All non-zero levels would be considered as positive levels. The inventors calculated the incidence of each species and combinations thereof. The inventors used Pearson correlation analysis to investigate the correlation between species level with age and gender. The correlation between species level and age was analyzed by Pearson correlation and the correlation between species level and gender was assessed by Spearman correlation.

### Results

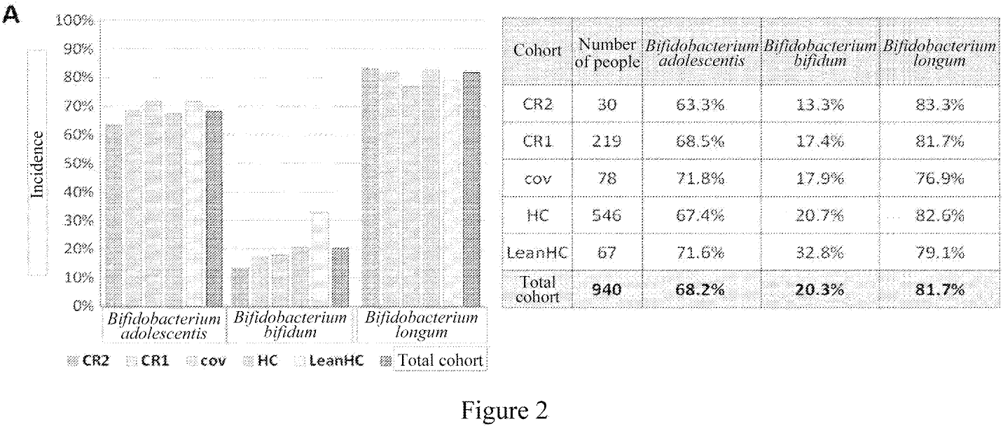

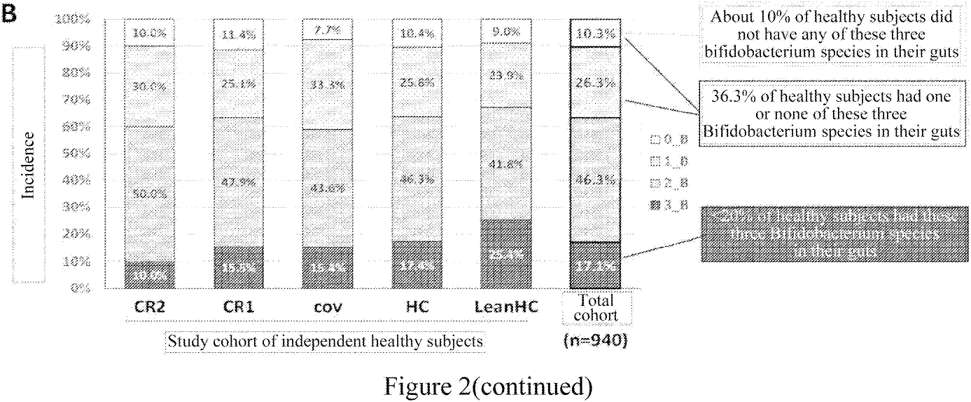

In the overall analysis results of the above healthy cohort, the positive rates of Bifidobacterium adolescentis, Bifidobacterium bifidum, and Bifidobacterium longum were 68.2%, 20.3%, and 81.7%, respectively (see FIG. 2, panel A). Less than 20% of healthy subjects had all of these three bifidobacterium species in their guts, 46.3% of healthy subjects had two of these three bifidobacterium species in their guts, and up to 36.3% of healthy subjects had only one or none of these three bifidobacterium species in their guts (see FIG. 2, panel B). The inventors further analyzed the correlation of three bifidobacterium species levels with age and gender. The results showed that age increase was negatively correlated with all of these three bifidobacteria. Male sex was significantly correlated with low level of Bifidobacterium longum (Table 2). These results suggested that the majority of the population was suitable for supplementing the prebiotic/prebiotic/synbiotic composition of the present application, in particular the older population and the male population.

TABLE 2

Results of correlation analysis of three bifidobacteria with age and gender in the total cohort

| | Correlation with age | | | Correlation with gender | | |
|---|---|---|---|---|---|---|
| Variable | r | 95% confidence interval | P | r | 95% confidence interval | P |
| Bifidobacterium adolescentis | -0.103 | -0.186, -0.020 | 0.016 | -0.017 | -0.101, 0.067 | 0.691 |
| Bifidobacterium bifidum | -0.186 | -0.266, -0.104 | < 0.0001 | -0.039 | -0.122, 0.046 | 0.368 |
| Bifidobacterium longum | -0.173 | -0.253, -0.091 | < 0.0001 | -0.144 | -0.226, -0.060 | 0.001 |

### Example 3

This example describes the amelioration of symptoms and modulation of immune response markers in hospitalized COVID-19 patients by administrating the synbiotic composition of the present application. It has been reported that coronavirus disease-2019 (COVID-19) caused by SARS-CoV-2 virus not only targets the lung, but also targets a number of other organs, including the gut. Gut microorganisms can modulate the immune response of a host, and therefore may affect the severity and prognosis of COVID-19 patients. The gut microbiome in COVID-19 patients becomes unbalanced, e.g., a decrease in symbionts, or an increase in opportunistic pathogens, which are all associated with the severity of COVID-19 and the shedding of SARS-CoV-2 virus in feces. Furthermore, it has been reported that when the content of probiotics producing short-chain fatty acids in fecal samples is high, the infectivity of SARS-CoV-2 is low, which highlights the potential beneficial effects of beneficial bacteria in combating SARS-CoV-2 infection. The therapeutic regimen of rebalancing the gut microbiome in COVID-19 patients has the potential to improve clinical effects.

The inventors expected that the synbiotic composition of the present application can improve the clinical symptoms of COVID-19 patients and therefore, a preliminary study was designed and conducted to evaluate the effects of such synbiotic composition on the clinical symptoms of COVID-19, blood immune markers, and fecal microbiome of hospitalized COVID-19 patients. The inventors compared these results with those of hospitalized COVID-19 patients (control group) who received standard treatment during the same time period. The synbiotic composition used in this study was formulated as follows.

The synbiotic composition comprised Bifidobacterium adolescentis, Bifidobacterium bifidum and Bifidobacterium longum as the probiotics, and xylooligosaccharide, galactooligosaccharide and corn dietary fiber as the prebiotics, wherein the ratio of the amounts of Bifidobacterium adolescentis, Bifidobacterium bifidum and Bifidobacterium longum calculated by colony forming units (CFUs) was controlled to be (0.75-1): 1: (0.75-1), and the total amount of the three bacteria was controlled to be about 2×10¹¹CFU, and the ratio of xylooligosaccharide, galactooligosaccharide and corn dietary fiber by weight was controlled to be (0.25-0.5): (2-4): (0.5-0.75), and the total amount of xylooligosaccharide, galactooligosaccharide and corn dietary fiber was controlled to be 1.2-1.5 g.

### Overview of Research

The inventors enrolled SARS-CoV-2-positive adult patients admitted to Prince of Wales Hospital between Aug. 13, 2020 and Oct. 9, 2020. The enrolled COVID-19 patients would receive the standard treatment or synbiotic composition of the present application within 48 hours of admission. The subject would receive the standard treatment or take a capsule of the synbiotic composition for 28 days.

The main results were a comprehensive assessment of the following three indicators: the use of a general symptom questionnaire to assess whether symptoms were relieved, whether respiratory support was required, and antibody production from the start of treatment to week 5.

COVID-19 related symptoms evaluated by questions 1 to 19 and 26 in the general symptom questionnaire (Table 3) included fever, respiratory symptoms and general symptoms, with a minimum score of 20 (normal), a maximum score of 80 (most severe symptoms), and a score of 20 for complete remission of symptoms. Gastrointestinal (GI) symptoms were evaluated by questions 20 to 25 of Table 3, with a score of 6 for complete remission of GI symptoms.

SARS-CoV-2 immunoglobulin G (IgG) antibodies were detected within 2 weeks after patients were admitted to hospital. The inventors performed a clinical symptom assessment of the patients every two days until the symptoms disappeared or the patients were discharged from the hospital. The inventors also included another group of patients who received the standard treatment at the same time in the study as a comparison. In all subjects, the inventors collected blood samples at baseline and 5 weeks after the subjects took the synbiotic composition, and detected immune response markers in plasma at baseline and week 5 in standard treatment group and synbiotic group by using the MILLIPLEX®MAP immunomultiplex assay. The inventors also collected feces and quality of life questionnaires at baseline, weeks 2, 4 and 5 after the subjects took the synbiotic composition, respectively.

TABLE 3

General Symptom Questionnaire How much did the following symptoms affect you today Please circle the most appropriate answer (1-4).

| | Not affected | little affected | Affected | Severely affected |
|---|---|---|---|---|
| 1. Cough | 1 | 2 | 3 | 4 |
| 2. Shortness of breath | 1 | 2 | 3 | 4 |
| 3. Sore throat | 1 | 2 | 3 | 4 |
| 4. Runny nose | 1 | 2 | 3 | 4 |
| 5. Breathing | 1 | 2 | 3 | 4 |
| 6. Fatigue | 1 | 2 | 3 | 4 |
| 7. Malaise | 1 | 2 | 3 | 4 |
| 8. Loss of smell or taste | 1 | 2 | 3 | 4 |
| 9. Headache | 1 | 2 | 3 | 4 |
| 10. Chills | 1 | 2 | 3 | 4 |
| 11. Fever, °C | 1 | 2 | 3 | 4 |
| 12. Chest pain | 1 | 2 | 3 | 4 |
| 13. Conjunctivitis | 1 | 2 | 3 | 4 |
| 14. Muscle pain | 1 | 2 | 3 | 4 |
| 15. Arthralgia | 1 | 2 | 3 | 4 |
| 16. Inability to walk | 1 | 2 | 3 | 4 |
| 17. Rash | 1 | 2 | 3 | 4 |
| 18. Overall discomfort | 1 | 2 | 3 | 4 |
| 19. Bleeding | 1 | 2 | 3 | 4 |
| 20. Anorexia | 1 | 2 | 3 | 4 |
| 21. Nausea | 1 | 2 | 3 | 4 |
| 22. Vomiting | 1 | 2 | 3 | 4 |
| 23. Abdominal pain | 1 | 2 | 3 | 4 |
| 24.Abdominal distension | 1 | 2 | 3 | 4 |
| 25. Diarrhea | 1 | 2 | 3 | 4 |
| 26. Other symptoms Specify: | 1 | 2 | 3 | 4 |

### Research Method

### Subject Recruitment

This study was approved by the Joint Chinese University of Hong Kong-New Territories East Cluster Clinical Research Ethics Committee (The Joint CUHK-NTEC CREC, CREC Ref. No: 2020.407), and was registered at Clinical Trial Register (NCT04581018). All subjects signed written informed consent. The enrolled subjects included 25 subjects in the synbiotic composition group and 30 subjects in the standard treatment (SC) group. The inventors recruited 18-year-old or older patients who were diagnosed with SARS-CoV-2 infection by reverse transcriptase polymerase chain reaction (PCR) test and were hospitalized in Wales Hospital of Hong Kong from Aug. 13, 2020 to Oct. 9, 2020. Subjects who were receiving intensive care or using a ventilator, were allergic or intolerant to an intervention product or components thereof, had a known history of endocarditis or active endocarditis, recently received CAPD or hemodialysis, or were pregnant at recruitment were excluded. Subjects suffering from any disease that would prevent oral administration of probiotics or increase the risks associated with probiotics were also excluded. These risks included, but were not limited to, inability to swallow or risk of aspiration without other administration methods (e.g., no G/J tube), known increased risk of infection due to immunosuppression, such as a history of organ or hematopoietic stem cell transplantation, neutropenia (ANC <500 cells/µl), or HIV and CD4 <200 cells/µl.

The subjects, who were hospitalized between August 2020 and October 2020, were assigned to the synbiotic composition group or the standard treatment group. The subjects, who were discharged from hospital since July 2020, were assigned to the standard treatment group. In this study, the subjects took the capsule of the synbiotic composition with food for 28 days as instructed and the clinical symptoms were evaluated every two days until the symptoms disappeared or they were discharged from the hospital. Blood samples were collected at baseline and 5 weeks after the subjects first took the synbiotic composition or enrollment. Fecal samples and questionnaires concerning quality of life were collected at baseline and weeks 2, 4 and 5 after the subjects first took the synbiotic composition or were enrolled. This study was conducted in accordance with the Declaration of Helsinki.

### Blood Immune Markers

After the whole blood sample was stood for 60 minutes, the plasma was collected by centrifugation at 1500x g without brake for 10 min at 4° C. The undiluted plasma was transferred to a 15 ml polypropylene conical tube and then divided and stored at -80° C. for subsequent research. The levels of plasma cytokines and chemokines were measured by using Custom Premix Human Cyto Panel A 47 Plex (Millipore , # HCYTA-60K-47C). All samples were detected at the first thawing.

### Results

This study included 25 COVID-19 patients taking the synbiotic composition and 30 patients receiving the standard treatment. The clinical characteristics of patients were shown in Table 3. There were no significant differences in age, comorbidities, baseline symptom scores, and disease severity between the two groups. At the time of diagnosis, the vital signs, hypoxemia and inflammatory markers were similar between the two groups (data not shown).

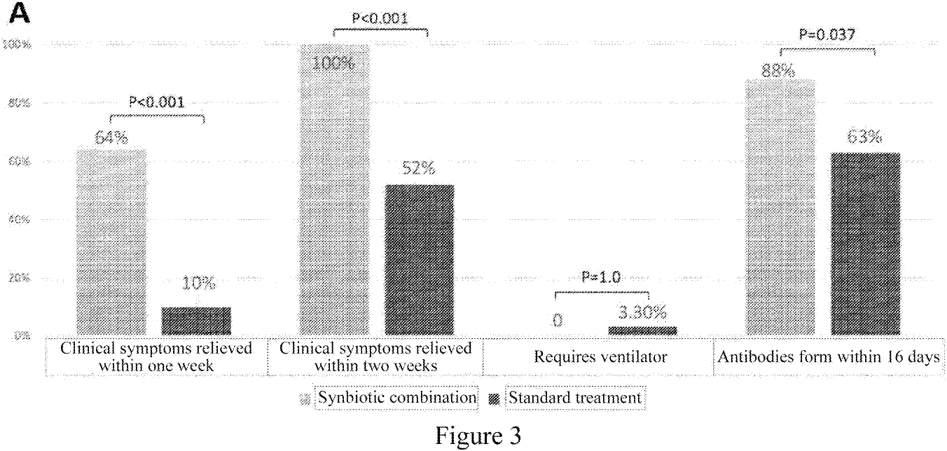

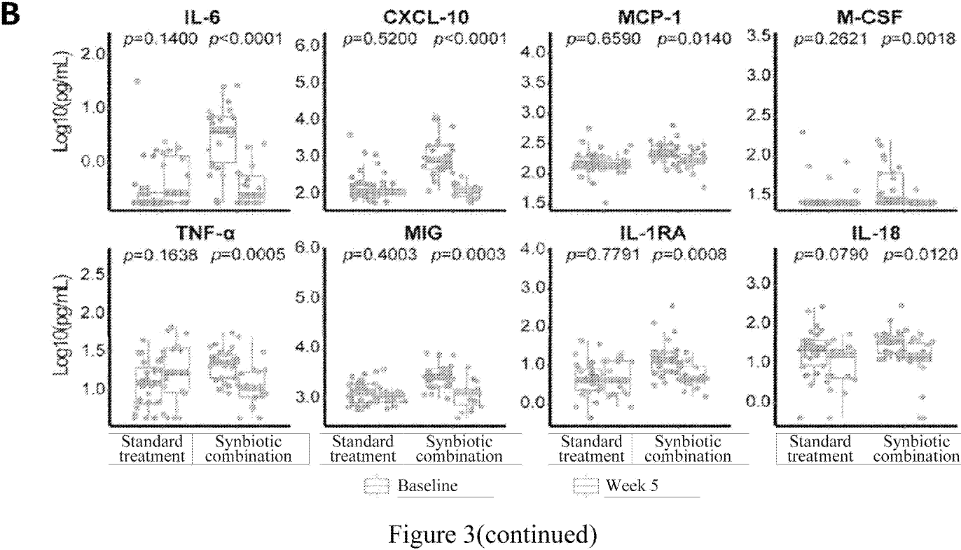

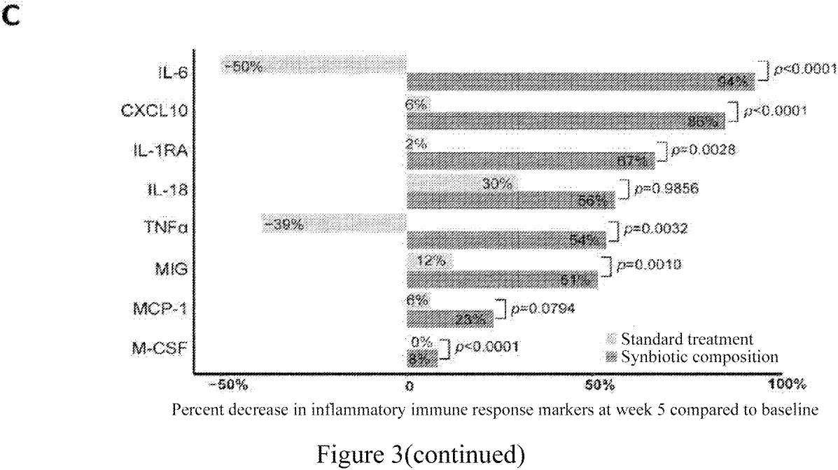

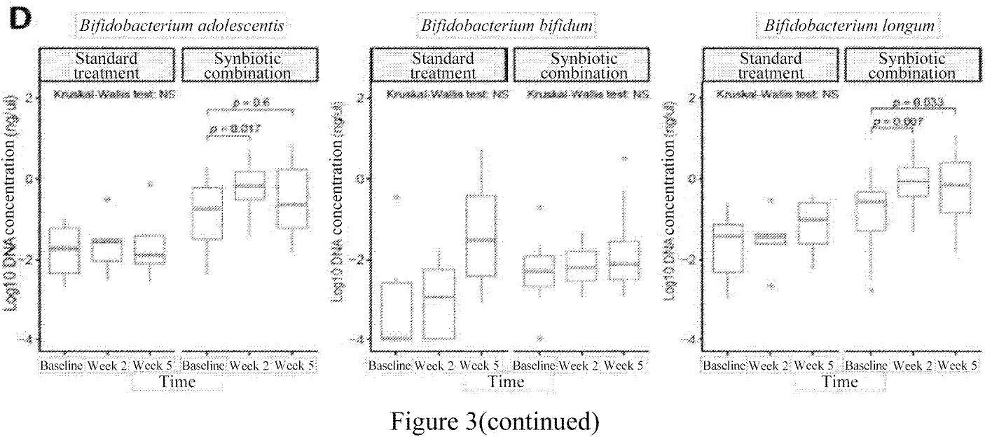

In the synbiotic composition group, the proportion of patients achieving complete symptom relief was significantly higher (FIG. 3, panel A, 64% vs 10% at week 1; p = <0.001; 100% vs 52% at week 2; p <0.001), and the antibody positive rate was also significantly higher compared to the standard treatment group (88% vs 63% on day 16; p = 0.037). Eight subjects (26.7%) in the standard treatment group and 1 subject (4%) in the synbiotic composition group were never positive for IgG antibody detection before discharge. Gastrointestinal (GI) symptoms of all patients receiving the synbiotic composition were relieved by week 2 (FIG. 3, panel A). The subjects in the synbiotic composition group had a significant improvement in quality of life at week 4 compared to baseline as assessed by the EuroQol Visual Analogue Scale (EQ-VAS) and the EuroQol Index Score (EQ Index Score) (specific data not shown) (EQ-VAS scores at week 4 and baseline were 81.5 and 69.75, respectively, p = 0.034; the EQ index scores at week 4 and baseline were 0.839 and 0.805, respectively, p <0.0005), but no such improvement was observed in the standard treatment group. There were no serious adverse reactions in both groups. In the synbiotic composition group, some adverse events were observed, including mild dizziness symptoms (4%), tinea infection (4%), and hypertension (4%). One subject (4%) was found to have chronic lymphocytic leukemia by a routine blood test. In the standard treatment group, 3 subjects (10%) suffered from constipation, and 1 subject (3.3%) suffered from mild hand inflammation. In the synbiotic composition group, the following eight major immune response markers at week 5 were significantly lower than their corresponding levels at baseline: interleukin (IL-6 , IL-1RA , IL-18), tumor necrosis factor (TNF-α), macrophage colony stimulating factor (M-CSF), CXC chemokine ligand 10 (CXCL-10, also referred to as IP10), monocyte chemoattractant protein 1 (MCP-1), and monokine induced by interferon-y (MIG). In contrast, in the standard treatment group, many of these immune response markers did not change significantly (FIG. 3, panel B). Compared to the baseline level, the percentage decrease of IL-6, CXCL-10, IL1RA, MIG, TNF-α and M-CSF at week 5 was significantly higher in the synbiotic composition group than in the control group (FIG. 3, panel C). In the synbiotic composition group, the abundance of Bifidobacterium adolescentis and Bifidobacterium longum at weeks 2 and 5 increased significantly compared to the baseline (FIG. 3, panel D).

TABLE 4

Patient characteristics of the synbiotic composition group and standard treatment group

| Characteristics | Synbiotic composition (n=25) | Standard treatment (n = 30) | P value |
|---|---|---|---|
| Male, n (%) | 14 (56) | 9 (30) | 0.052 |
| Median age, (IQR) years | 50 (39-59) | 46.5 (29.5- 56) | 0.151 |
| Smokers/ex-smokers, n (%) | 7 (28) | 6 (20) | 0.298 |
| Drinkers/abstainers, n (%) | 2 (8) | 1 (3) | 0.585 |
| Comorbidities, n (%) | 11 (44) | 16 (53) | 0.729 |
| Recent exposure history, n (%) | | | |
| Travel history | 0(0) | 2 (6.7) | 1.000 |
| Exposure to COVID19 patients | 18 (72) | 19 (63) | 0.448 |
| Admission symptoms, n (%) | | | |
| Fever | 20 (80) | 15 (50) | 0.030 |
| Loss of taste/smell | 4(16) | 4 (13) | |
| Myalgia | 5(20) | 1 (3) | 0.085 |
| Malaise | 3(12) | 7 (23) | 0.309 |
| Gastrointestinal symptoms | | | |
| Diarrhea | 6(24) | 5 (17) | 0.736 |
| Respiratory symptoms | | | |
| Cough | 15 (60) | 16(53) | 0.721 |
| Phlegm | 3 (12) | 8 (27) | 0.156 |
| Sore throat | 7 (28) | 11 (37) | 0.440 |
| Rhinorrhea | 4 (16) | 5 (17) | 1.000 |
| Shortness of breath | 4 (16) | 3 (10) | 0.692 |
| Median of symptoms at baseline (IQR) | 25 (22-30) | 25.5 (22-29) | 0.575 |
| Drugs against COVID-19, n (%) | | | |
| Antibiotics | 4(16) | 6 (20) | 0.741 |
| Antiviral drugs | 12 (48) | 19(63) | 0.254 |
| Dexamethasone | 4(16) | 5(17) | 1.000 |
| Chest X-ray examination at admission, n (%) | | | |
| infiltration | 10 (40) | 13 (43) | 1.000 |
| Severity at admission, n (%) | | | |
| Mild | 16 (64) | 21 (70) | 0.637 |
| Medium | 8 (32) | 8 (27) | 0.665 |
| Severe | 1 (4) | 1 (3) | 1.000 |
| Critical | 0 (0) | 0(0) | N/A |

IQR: Median of symptoms at baseline

### Discussion

This preliminary study found that the use of the synbiotic formulation of the present application can alleviate gastrointestinal symptoms and inhibit inflammatory cytokine levels in hospitalized COVID-19 patients. The theoretical basis of this study is that the use of the synbiotic composition in COVID-19 patients can rebalance the gut microbiome, thereby reducing the severity of the disease and improving the quality of life. Because the anti-SARS-CoV-2 effects of synbiotics have not yet been studied in COVID-19 patients before this study, relevant pre-event data are limited.

The inventors have discovered for the first time that the guts in COVID-19 patients lack a series of beneficial bacteria and the activities of viral infection and replication are still maintained in the gut after SARS-CoV-2 virus is cleared in the respiratory tract. By using large data analysis and machine learning, the inventors developed a probiotic formulation for gut microecological imbalance. In the synbiotic composition group, the abundance of probiotics increased significantly at week 2, confirming that the probiotics had been successfully delivered to the gut. Studies showed elevated levels of immune response markers in COVID-19 critically ill patients, and these markers include IL-6, IL-1RA, IL-18, TNF-α, M-CSF, CXCL10, MCP-1, and MIG. The inventors have discovered that the synbiotic composition can reduce the levels of the above eight immune response markers in plasma samples at week 5. Meanwhile, the COVID-19 symptoms of the patients were relieved at weeks 2 and 5 after treatment with the symbiotic composition. These results suggest that the treatment with the synbiotic composition may enhance the host immune response to SARS-CoV2, primarily reflected by suppressing cytokines that increase in the early stage of COVID-19 infection. The results provide a basis for targeted therapy of synbiotics against gut flora. The inventors’ study proposes that the synbiotics have an effect of early immune intervention, which provides reference and hope for the application of the synbiotic composition to enhance the immunity of organisms against COVID-19 and other emerging viral infections.

### Example 4

This example is an extension study based on Example 3. 25 COVID-19 patients taking the synbiotic composition and 10 patients receiving the standard treatment from Example 3 were included. Additionally, 69 patients receiving the standard treatment and 78 healthy people as a control group were included. By meta-genomic analysis, it was found that the synbiotic formulation of the present application can restore the unbalanced gut microecology to balance and reach or approach levels in normal humans.

### Research Method

### Subject Recruitment

The inventors recruited 25 COVID-19 patients taking the synbiotic composition and 10 patients receiving the standard treatment from Example 3. Additionally, the inventors recruited 69 patients receiving the standard treatment. This study was approved by the Joint Chinese University of Hong Kong-New Territories East Cluster Clinical Research Ethics Committee (The Joint CUHK-NTEC CREC, CREC Ref. No: 2020.076) and was conducted in accordance with the Declaration of Helsinki. All subjects signed written informed consent. These COVID-9 patients were recruited from Prince of Wales Hospital and United Christian Hospital in Hong Kong between February 2020 and May 2020. The nasopharyngeal swabs of these patients were collected by hospital staff and were confirmed to be positive for SARS-CoV-2 by quantitative reverse transcription polymerase chain reaction (RT-qPCR) in the laboratory. The inventors also recruited 78 healthy people as a control group from a survey of gut microbiome of Hong Kong’s population. These healthy people were recruited by advertising or colonoscopy.

### Fecal Sample Collection

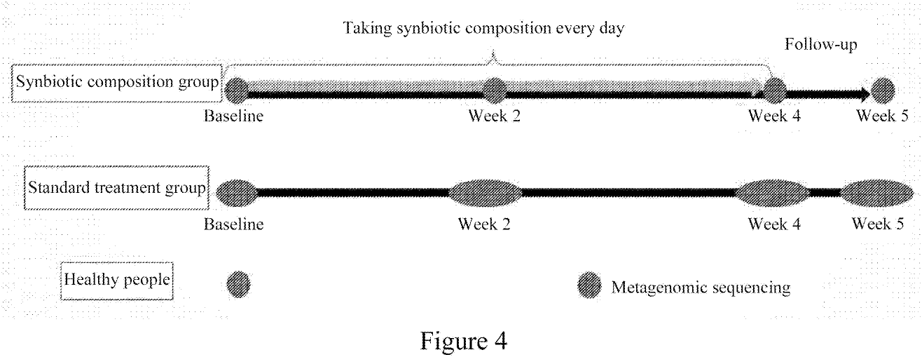

As described in FIG. 4, the inventors collected fecal samples from subjects in the synbiotic composition and standard treatment group at different time points (baseline, weeks 2, 4, and 5). Additionally, the inventors collected fetal samples from the healthy control group at a single time point.

### Fecal Samples and DNA Extraction

Fecal DNA was extracted by using QIAamp DNeasy PowerSoil Kit, Maxwell RSC PureFood GMO and Authentication Kit, or QIAamp DNA Stool Mini Kit according to the manufacturer’s instructions. The quality and quantity of DNA were determined by using a NanoDrop spectrophotometer and gel electrophoresis, respectively.

### Meta-Genome Sequencing

A DNA library was constructed by end repair, purification and PCR amplification procedures. After constructing the DNA library, the DNA library was sequenced on the NextSeq platform in the inventors’ laboratory using the 150 bp paired-end sequencing strategy. An average of 12 Gb of data per sample was available for further analysis. All experimental procedures were in accordance with the uniform standards of the inventors’ laboratory.

### Data Processing and Statistical Analysis of Metagenomic Data

Fastp was used to perform mass filtration, PolyG tail modification and adapter modification on the sequenced fragments of metagenome, and the sequenced fragments of 50 bases or less were deleted. Next, the human genes in the sequenced fragments of the quality-trimmed metagenome were removed with KneadData, and then the metagenome at species level was analyzed with MetaPhlAn 2. All non-zero levels would be considered as positive levels. The inventors calculated the incidence of each species and combinations thereof. As described in Example 1, according to the inventors’ previous research results, the species, in which a variety of bacteria including a plurality of Bacteroides and Bifidobacterium pseudocatenulatum in the gut were negatively correlated with the disease severity or SARS-CoV-2 viral load of COVID-19 patients, were defined as “desirable bacterial species” (related studies were described in U.S. Provisional Patent Applications 63/016,759 and 63/025,310). Additionally, the species with a relatively high abundance in the gut of COVID-19 patients were defined as “undesirable bacterial species”. The a biodiversity index (Shannon Diversity Index) was calculated by using the Vegan package in the R software. Differences between the microbiome were analyzed by using LEfSe software (linear discriminant analysis [LDA] effect size). Species with LDA greater than 2 and p < 0.05 were considered to have significant differences between groups.

### Results

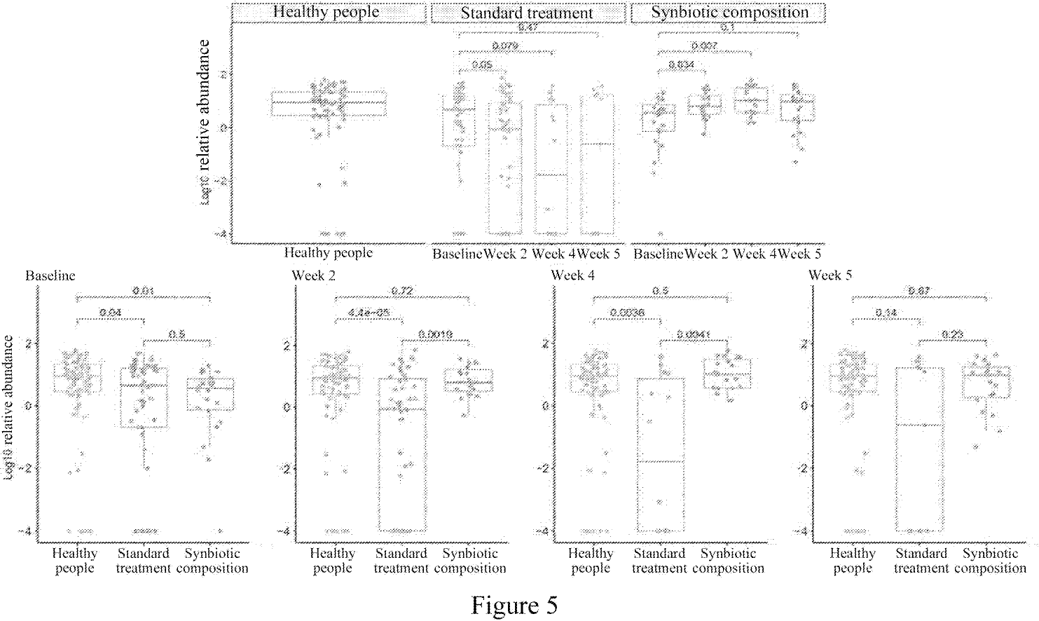

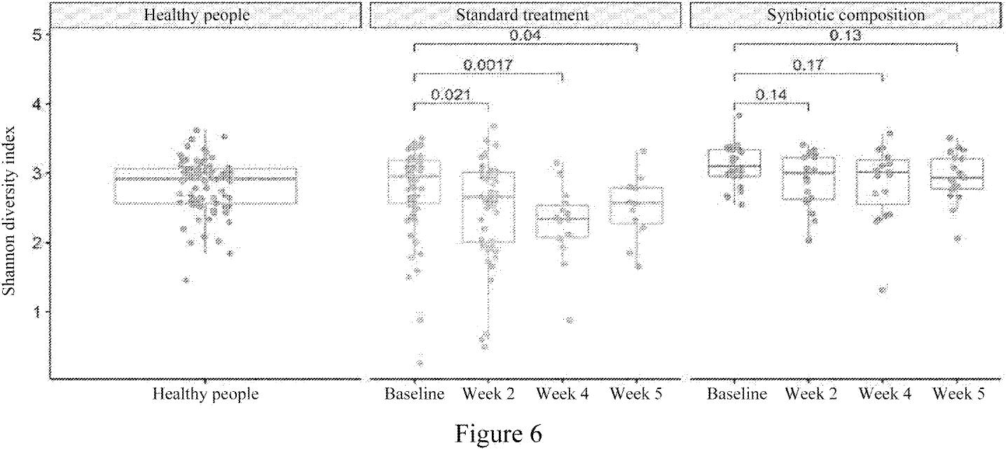

In COVID-19 patients receiving the standard treatment, the total abundance of the three bifidobacterium probiotics in the synbiotic formulation of the present application decreased slightly at weeks 2 and 4 compared with the baseline. In contrast, in patients receiving the synbiotic composition, the total abundance of the three bifidobacterium probiotics at weeks 2 and 4 increased significantly (FIG. 5). Compared with the baseline, the Shannon diversity index in COVID-19 patients receiving the standard treatment significantly decreased at weeks 2, 4 and 5; and in contrast, the Shannon diversity index was maintained at a high level similar to baseline in COVID-19 patients receiving the synbiotic composition (FIG. 6).

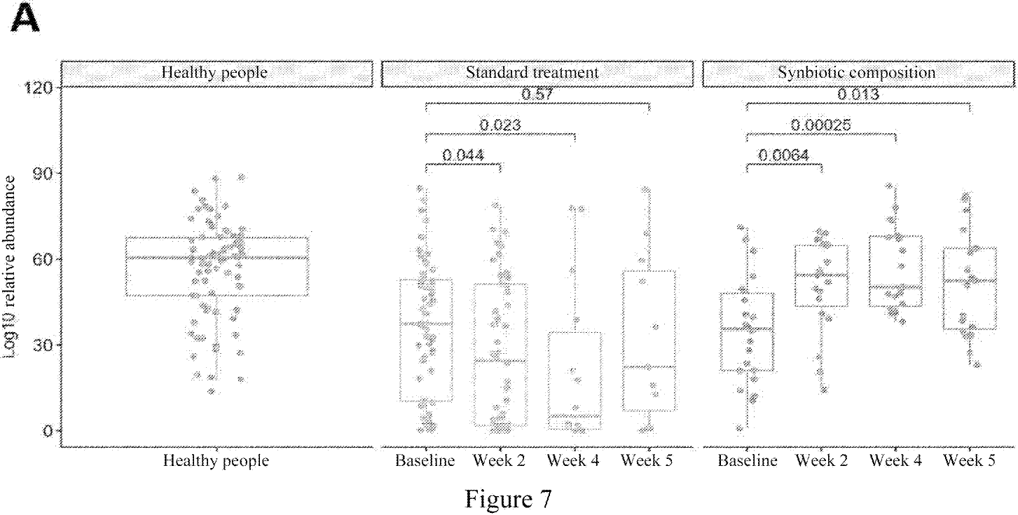

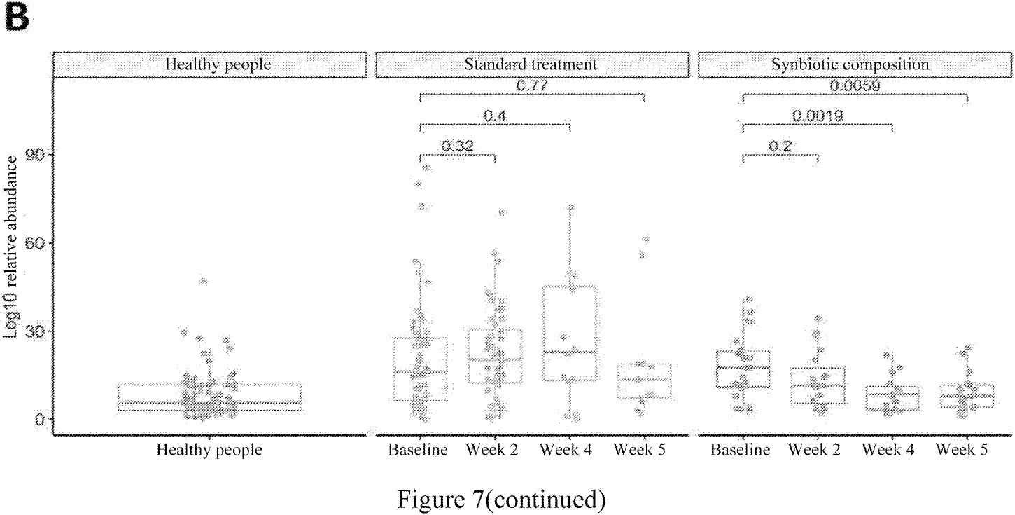

The total richness of the desirable bacterial species in COVID-19 patients receiving the standard treatment was significantly reduced at weeks 2 and 4 compared with the baseline. In contrast, the total richness of the desirable bacterial species in COVID-19 patients receiving the synbiotic composition was significantly increased at weeks 2 and 4 compared with the baseline (FIG. 7, panel A). Meanwhile, compared with the baseline, the total richness of the undesirable bacterial species in COVID-19 patients receiving the synbiotic composition was significantly reduced at weeks 2, 4, and 5, but not in patients receiving the standard treatment (FIG. 7, panel B).

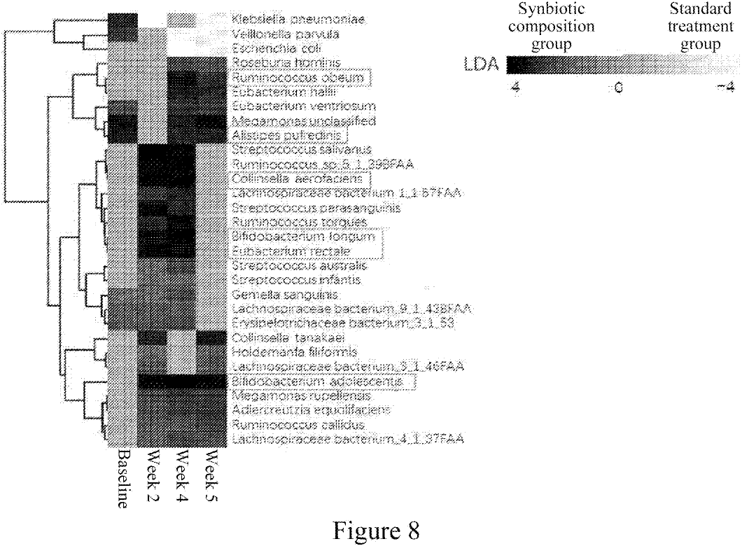

In addition, the desirable bacterial species including Bifidobacterium adolescentis, Eubacterium rectale, Ruminococcus, and Bifidobacterium longum in COVID-19 patients receiving the synbiotic composition were significantly richer than in COVID-19 patients receiving the standard treatment (FIG. 8).

The foregoing has described exemplary embodiments of the present application, but those skilled in the art can alter or modify the exemplary embodiments described in the present application, thereby obtaining variations or equivalents thereof, without departing from the spirit and scope of the present application.

What is claimed is:

1. A probiotic composition comprising Bifidobacterium bifidum and Bifidobacterium longum.

2. The probiotic composition according to claim 1, wherein the ratio of the amounts of Bifidobacterium bifidum and Bifidobacterium longum calculated by colony forming units is 1: (0.21-2.36).

3. The probiotic composition according to claim 1 or 2, further comprising Bifidobacterium adolescentis.

4. The probiotic composition according to claim 3, wherein the ratio of the amounts of Bifidobacterium adolescentis, Bifidobacterium bifidum and Bifidobacterium longum calculated by colony forming units is (0.57-3.56): 1: (0.21-2.36), preferably (0.75-1): 1: (0.75-1).

5. The probiotic composition according to any one of claims 1 to 4, further comprising Lactobacillus rhamnosus.

6. The probiotic composition according to claim 5, wherein the ratio of the amounts of Bifidobacterium adolescentis, Bifidobacterium bifidum, Bifidobacterium longum, and Lactobacillus rhamnosus calculated by colony forming units is (0.57-3.56): 1: (0.21-2.36): 1.

7. The probiotic composition according to any one of claims 1 to 6, wherein the probiotic composition is in unit dosage form, and the amounts of Bifidobacterium adolescentis, Bifidobacterium bifidum, Bifidobacterium longum, Lactobacillus rhamnosus calculated by colony forming units are independently in the order of 10⁴ to 10¹² CFU; optionally, the total amount of Bifidobacterium adolescentis, Bifidobacterium bifidum, Bifidobacterium longum, Lactobacillus rhamnosus is in the order of 10⁶ to 10¹² CFU.

8. The probiotic composition according to claim 7, wherein the probiotic composition is for administration to an adult, and
the amount of Bifidobacterium adolescentis is 2.59x10⁵-4.49x10¹¹ CFU; and/or
the amount of Bifidobacterium bifidum is 1.26x10⁵-7.35x10¹¹ CFU; and/or
the amount of Bifidobacterium longum is 2.23x10⁵-7.02x10¹¹ CFU; and/or
the amount of Lactobacillus rhamnosus is 1.26x10⁵-2.59x10¹¹ CFU.

9. The probiotic composition according to claim 7, wherein the probiotic composition is for administration to a child, and
the amount of Bifidobacterium adolescentis is 2.05x10⁵-4.55x10¹¹ CFU; and/or
the amount of Bifidobacterium bifidum is 1.47x10⁵-3.6x10¹¹ CFU; and/or
the amount of Bifidobacterium longum is 7.55x10⁴- 2.5x10¹¹ CFU; and/or
the amount of Lactobacillus rhamnosus is 1.47x10⁵-3.6x10¹¹ CFU.

10. The probiotic composition according to any one of claims 1 to 9, wherein the probiotic composition is free of probiotics other than Bifidobacterium adolescentis, Bifidobacterium bifidum, Bifidobacterium longum and Lactobacillus rhamnosus, and for example, the probiotic composition is free of Bifidobacteria other than Bifidobacterium adolescentis, Bifidobacterium bifidum and Bifidobacterium longum.

11. A prebiotic composition comprising xylooligosaccharide, galactooligosaccharide and corn dietary fiber.

12. The prebiotic composition according to claim 11, wherein the ratio of the amounts of xylooligosaccharide, galactooligosaccharides and corn dietary fiber by weight is (0.25-5): (0.75-4): (0.5-1), preferably (0.25-0.5): (2- 4): (0.5-0.75).

13. The prebiotic composition according to claim 11 or 12, wherein the prebiotic composition is in unit dosage form, and the total amount of the xylooligosaccharide, galactooligosaccharide and corn dietary fiber by weight is 0.1-12 g, such as 0.1-5 g.

14. The prebiotic composition of claim 13, wherein
the amount of xylooligosaccharide is 0.01 g-6 g; and/or
the amount of galactooligosaccharide is 0.04 g-9.6 g; and/or
the amount of corn dietary fiber is 0.01 g-6g.

15. The prebiotic composition according to any one of claims 11 to 14, which is free of prebiotic components other than xylooligosaccharide, galactooligosaccharide and corn dietary fiber.

16. A dietary composition, comprising the probiotic composition of any one of claims 1 to 10 and the prebiotic composition of any one of claims 11 to 15; preferably, the dietary composition comprises Bifidobacterium adolescentis, Bifidobacterium bifidum and Bifidobacterium longum as the probiotics, and xylooligosaccharide, galactooligosaccharide and corn dietary fiber as the prebiotics, wherein the ratio of the amounts of Bifidobacterium adolescentis, Bifidobacterium bifidum and Bifidobacterium longum calculated by colony forming units (CFUs) is (0.75-1): 1: (0.75-1), and the total amount of the three bacteria is about 2x10¹¹CFU, and the ratio of xylooligosaccharide, galactooligosaccharide and corn dietary fiber by weight is (0.25-0.5): (2-4): (0.5-0.75), and the total amount of xylooligosaccharide, galactooligosaccharide and corn dietary fiber is 1.2-1.5 g.

17. The probiotic composition according to any one of claims 1 to 10, or the prebiotic composition according to any one of claims 11 to 15, or the dietary composition according to claim 16, which is formulated for peroral administration, such as oral administration, mixing with oral products, or gavage.

18. The probiotic composition according to any one of claims 1 to 10 and 17, or the prebiotic composition according to any one of claims 11 to 15 and 17, or the dietary composition according to claim 16 or 17, which is a food supplement, a food additive or a food.

19. The probiotic composition according to any one of claims 1 to 10, 17 and 18, or the prebiotic composition according to any one of claims 11 to 15, 17 and 18, or the dietary composition according to any one of claims 16 to 18, which is formulated into a powder, a granule, a tablet or a capsule.

20. A probiotic composition according to any one of claims 1 to 10 and 17 to 19, or a prebiotic composition according to any one of claims 11 to 15 and 17 to 19, or the dietary composition according to any one of claims 16 to 19, which is administered to a subject for assisting in preventing and/or treating a pathogen infection, or for enhancing the therapeutic effect of a pathogen infection, improving the immunity in the subject, or balancing the gut microecology in the subject (including increasing microbial abundance, increasing desirable bacterial species, and/or reducing undesirable bacteria).

21. The probiotic composition, or prebiotic composition, or dietary composition according to claim 20, wherein the pathogen is a virus, a bacterium, or a fungus, such as a respiratory disease virus, such as a novel coronavirus (COVID-19), an influenza virus, or a respiratory syncytial virus.

22. Use of the probiotic composition according to any one of claims 1 to 10 and 17 to 19, or the prebiotic composition according to any one of claims 11 to 15 and 17 to 19, or the dietary composition according to any one of claims 16 to 19, in the preparation of a dietary product or a drug for assisting in preventing and/or treating the pathogen infection in a subject, enhancing the therapeutic effect of a pathogen infection in a subject, or improving the immunity in a subject, or balancing the gut microecology in a subject (including increasing microbial abundance, increasing desirable bacterial species, and/or reducing undesirable bacteria).

23. A method for assisting in preventing and/or treating a pathogen infection in a subject, enhancing the therapeutic effect of a pathogen infection in a subject, or improving the immunity in a subject, or balancing the gut microecology in a subject (including increasing microbial abundance, increasing desirable bacterial species, and/or reducing undesirable bacteria), comprising administering to the subject the probiotic composition according to any one of claims 1 to 10 and 17 to 19, or the prebiotic composition according to any one of claims 11 to 15 and 17 to 19, or the dietary composition according to any one of claims 16 to 19.
